(19) United States
(12) Patent Application Publication
(10) Pub. No.: US 2024/0116046 A1
(43) Pub. Date: Apr. 11, 2024

(54) SENSOR PACKAGE, SENSOR MODULE, AND SENSOR DEVICE

(71) Applicant: KYOCERA Corporation, Kyoto-shi, Kyoto (JP)

(72) Inventors: Hisashi SAKAI, Kyoto-shi, Kyoto (JP); Masahiko TAJIMA, Kyoto-shi, Kyoto (JP); Tadatomo MAEHARA, Kyoto-shi, Kyoto (JP); Masami YOSHIKAWA, Kyoto-shi, Kyoto (JP)

(21) Appl. No.: 18/276,854

(22) PCT Filed: Feb. 9, 2022
(86) PCT No.: PCT/JP2022/005139

§ 371 Date: Aug. 10, 2023

(30) Foreign Application Priority Data
Feb. 17, 2021 (JP) 2021-023740

(51) Int. Cl. B01L 3/00

(52) U.S. Cl. CPC B01L 3/502; B01L 2200/025; B01L 2200/0689; B01L 2300/0645; B01L 2300/0663

(57) ABSTRACT

A sensor package 10 includes a plurality of sensors 19 and a container 18. Each of the sensors 19 detects a detection target component in the fluid. The container 18 includes an inner channel 21 and a first surface os1. The plurality of sensors 19 are provided in the inner channel 21. The inner channel 21 enables the fluid to flow. An inflow opening 25 to the inner channel 21 and an outflow opening 25 from the inner channel 21 are formed on the first surface os1. An electrode group electrically connected to the plurality of sensors 19 is provided on a surface different from the first surface os1 of the container 18.

### CROSS-REFERENCE TO RELATED APPLICATIONS

This application claims the priority of Japanese Patent Application No. 2021-023740 filed on Feb. 17, 2021, and the entire disclosure of the earlier application is incorporated herein by reference.

### TECHNICAL FIELD

The present disclosure relates to a sensor package, a sensor module, and a sensor device.

### BACKGROUND OF INVENTION

A measuring device is known in which a crystal resonator functioning as a sensor is disposed in a channel pipe in order to detect odor in a space (see Patent Document 1). Odor is perceived by an organism as a single molecule or a group of molecules composed of a plurality of different molecules, and the usage of a plurality of sensors to detect odor is known (see Patent Document 2).

### CITATION LIST

### Patent Literature

Patent Document 1: JP 2012-2691 A
Patent Document 2: WO 2018/211642

### SUMMARY

A sensor package according to a first aspect includes:
a plurality of sensors configured to detect a detection target component in a fluid; and
a container including an inner channel in which the plurality of sensors are provided and through which a fluid flows, and a first surface on which an inflow opening to the inner channel and an outflow opening from the inner channel are formed, wherein
an electrode group electrically connected to the plurality of sensors is provided on a surface different from the first surface of the container.

A sensor module according to a second aspect includes:
a package placement surface on which a supply port and a discharge port configured to enable a fluid to flow to a sensor package are provided, the sensor package including a plurality of sensors configured to detect a detection target component in a fluid; and a container including an inner channel in which the plurality of sensors are provided and through which a fluid flows, and a first surface on which an inflow opening to the inner channel and an outflow opening from the inner channel are formed, an electrode group electrically connected to the plurality of sensors being provided on a surface different from the first surface of the container;
a sealing body provided at the supply port and the discharge port and configured to seal a connecting portion between the supply port and the inflow opening and a connecting portion between the discharge port and the outflow opening; and
a fixing portion configured to detachably fix the sensor package thereby pressing the sensor package against the package placement surface.

A sensor device according to a third aspect includes:
a sensor module including:
a package placement surface on which a supply port and a discharge port configured to enable a fluid to flow to a sensor package are provided, the sensor package including a plurality of sensors configured to detect a detection target component in a fluid; and a container including an inner channel in which the plurality of sensors are provided and through which a fluid flows, and a first surface on which an inflow opening to the inner channel and an outflow opening from the inner channel are formed, an electrode group electrically connected to the plurality of sensors being provided on a surface different from the first surface of the container;
sealing bodies respectively provided at the supply port and the discharge port and configured to seal a connecting portion between the supply port and the inflow opening and a connecting portion between the discharge port and the outflow opening; and
a fixing portion configured to detachably fix the sensor package thereby pressing the sensor package against the package placement surface.

### BRIEF DESCRIPTION OF THE DRAWINGS

FIG. 1 is a schematic view of a sensor module according to the present embodiment.

FIG. 2 is a perspective view of a sensor package in FIG. 1 cut along a surface perpendicular to a second direction.

FIG. 3 is a cross-sectional view of the main portion in FIG. 2 taken along a surface perpendicular to a first direction.

FIG. 4 is a perspective view illustrating an inner channel in FIG. 2.

FIG. 5 is a perspective view of a sensor package viewed from the normal direction of the bottom surface, illustrating the inner channel in FIG. 2.

FIG. 6 is a perspective view illustrating a variation of the inner channel in FIG. 2.

FIG. 7 is a top view of the sensor package in FIG. 1.

FIG. 8 is an external view illustrating a state in which a flexible substrate is connected to the sensor package in FIG. 1.

FIG. 9 is an external view illustrating a back surface of the sensor package in FIG. 8.

FIG. 10 is a cross-sectional view of a socket that can be mated with the sensor package in FIG. 1.

FIG. 11 is a perspective view illustrating an outer appearance of a sensor in FIG. 2.

FIG. 12 is a cross-sectional view of the sensor module in FIG. 2 taken along a surface perpendicular to the first direction.

FIG. 13 is an external view of a sensor module to which the sensor package in FIG. 1 is fixed.

FIG. 14 is a partial external view of a sensor module from which the sensor package in FIG. 1 is removed.

FIG. 15 is a cross-sectional view taken along a line XV-XV in FIG. 13.

FIG. 16 is an external view illustrating a state in which the sensor package is mounted on a package placement surface in a normal position and a normal posture in FIG. 14.

FIG. 17 is a cross-sectional view of a variation of the sensor package.

FIG. 18 is a functional block diagram illustrating an overall configuration of the sensor module in FIG. 1.

FIG. 19 is a diagram schematically illustrating an example of fluid flow.

FIG. 20 is a diagram schematically illustrating an example of fluid flow.

### DESCRIPTION OF EMBODIMENTS

Embodiments according to the present disclosure will be described below with reference to the drawings.

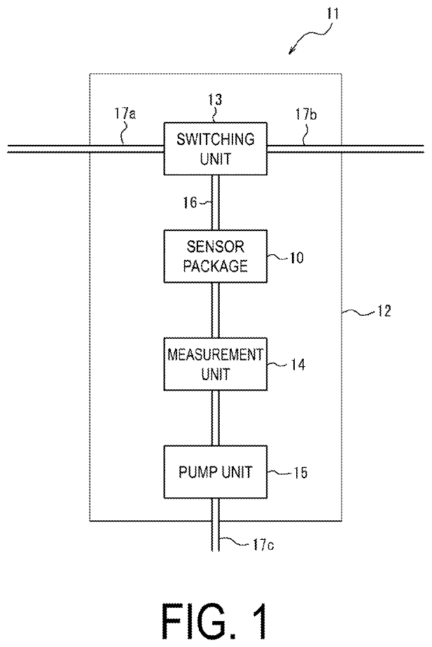

FIG. 1 is a schematic view of a sensor module 11 including a sensor package 10 according to an embodiment of the present disclosure. The sensor module 11 may be incorporated into a sensor device, for example.

The sensor module 11 includes, for example, a housing 12. The housing 12 may house each functional unit included in the sensor module 11. The sensor module 11 may be supplied with a fluid. The sensor module 11 may be able to calculate a concentration of a first component, which is a detection target component contained in a test fluid, based on a fluid to be tested (test fluid) and a fluid to be compared (control fluid). Hereinafter, the side to which fluid is supplied is also referred to as an upstream side, and the side from which fluid is discharged is also referred to as a downstream side.

The sensor module 11 may include a switching unit 13, the sensor package 10, a measurement unit 14, and a pump unit 15 inside the housing 12. In the sensor module 11, the switching unit 13, the sensor package 10, the measurement unit 14, and the pump unit 15 may be disposed in this order from the upstream side in one channel 16. The channel 16 may be constituted by a pipe-shaped member such as a tube. A first channel 17a and a second channel 17b may be further connected to the upstream side of the switching unit 13. Fluid may be supplied to the inside of the sensor module 11 from the first channel 17a and the second channel 17b, and the fluid may be discharged to the outside from a third channel 17c connected to the downstream side of the pump unit 15.

The test fluid may be supplied to the first channel 17a. The control fluid may be supplied to the second channel 17b. An exhaust fluid may be exhausted to the third channel 17c. The first channel 17a, the second channel 17b, and the third channel 17c may be constituted by a pipe-shaped member such as a tube.

The switching unit 13 may selectively switch an open/closed state of the first channel 17a and the second channel 17b. That is, the switching unit 13 can selectively connect one of the first channel 17a and the second channel 17b to the channel 16. Therefore, when the first channel 17a is connected to the channel 16 by the switching unit 13, the second channel 17b is not connected to the channel 16. In this case, the test fluid is supplied to the channel 16 via the first channel 17a. On the other hand, when the second channel 17b is connected to the channel 16 by the switching unit 13, the first channel 17a is not connected to the channel 16. In this case, the control fluid is supplied to the channel 16 via the second channel 17b. The switching unit 13 may be configured to include a valve capable of switching between the first channel 17a and the second channel 17b, for example.

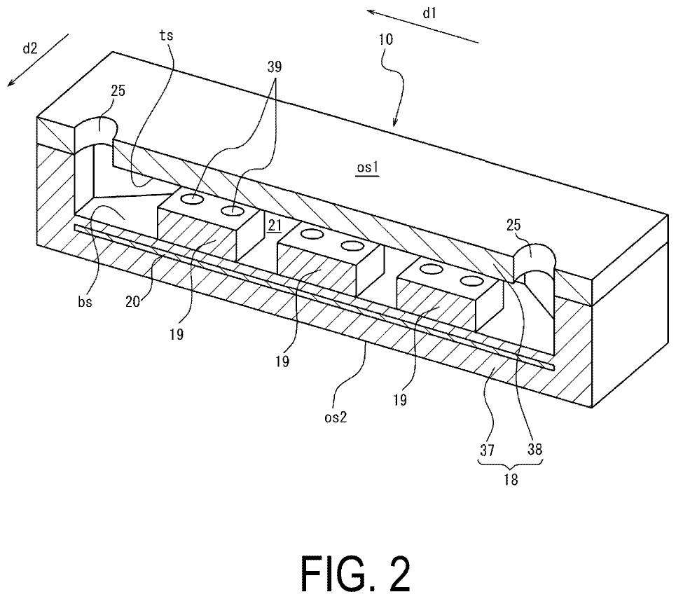

As illustrated in FIG. 2, the sensor package 10 includes a container 18 and a plurality of sensors 19. The sensor package 10 may further include a heater 20.

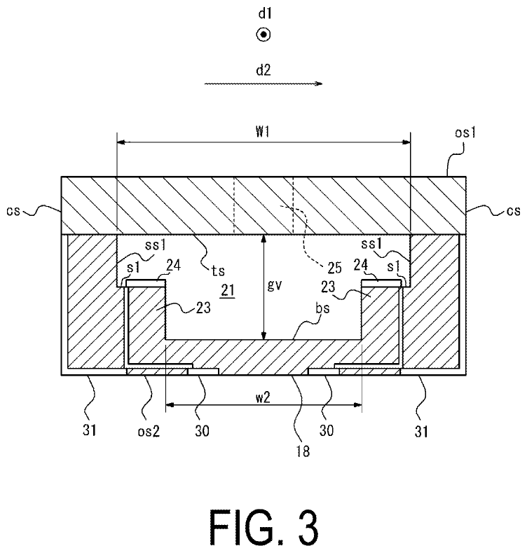

The container 18 includes a first surface (first face) os1. The first surface os1 may be a flat surface or a curved surface. As illustrated in FIG. 3, the container 18 may further include a second surface (back surface) os2 and a container side surface cs. The second surface os2 may be a back surface of the first surface os1. The container side surface cs may be a surface between the first surface os1 and the second surface os2, for example a surface connecting the first surface os1 and the second surface os2. The container side surface cs may be a surface extending in the first direction d1. The second surface os2 and the container side surface cs may be flat surfaces or curved surfaces. The container 18 may have a rectangular parallelepiped shape.

The container 18 may be made of ceramic, plastic, metal, or the like. In the present embodiment, when the container 18 is made of ceramic, adsorption of the fluid and degassing from the container 18 can be suppressed.

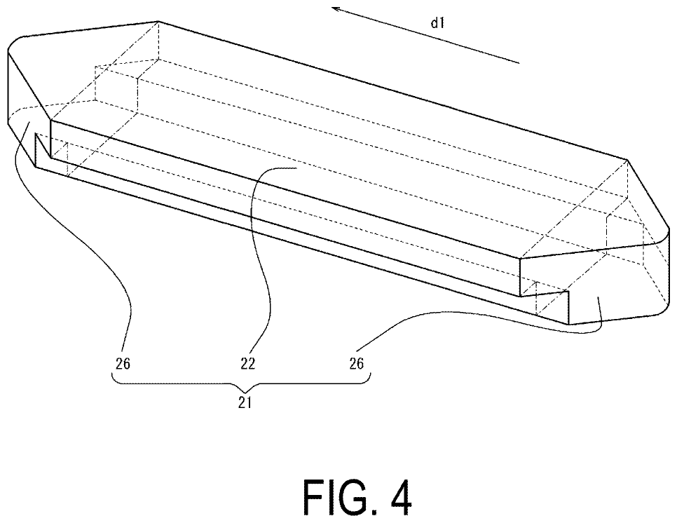

The container 18 includes an inner channel 21 therein. The inner channel 21 includes the plurality of sensors 19. The inner channel 21 allows the fluid to flow. The inner channel 21 may cause the fluid to flow along the linear first direction d1. As illustrated in FIG. 4, the inner channel 21 may for example include a main portion 22 defined by a cylindrical inner wall extending along the first direction d1. As illustrated in FIG. 3, a part of the inner channel 21 may be defined by, for example, a flat bottom surface bs. A part of the inner channel 21 may be defined by, for example, a flat top surface ts facing the bottom surface bs.

A distance gv between the bottom surface bs and the top surface ts may be 1.5 times or more and 3 times or less the height of each of the sensors 19 to be described later. When the distance gv is 1.5 times or more, a space in which the fluid sufficiently flows is secured. When the distance gv is 1.5 times or more, the pressure distribution is made uniform and the output of the sensor 19 is stabilized. When the distance gv is three times or less, an unnecessary increase in the size of the sensor package 10 is suppressed. When the distance gv is 3 times or less, a decrease in the flow rate, the retention of the fluid, or the like can be reduced. In the present embodiment, the distance gv between the bottom surface bs and the top surface ts is twice the height of the sensor 19. Therefore, in the present embodiment, the distance between each of the sensors 19 fixed to the bottom surface bs and the top surface ts is the same as the height of each of the sensors 19.

As illustrated in FIG. 3, a part of the main portion 22 may be defined by side surfaces ss1 perpendicular to the bottom surface bs and parallel to the first direction d1. The side surfaces ss1 may be connected to the bottom surface bs at both ends of the bottom surface bs in a second direction d2 parallel to the bottom surface bs and perpendicular to the first direction d1. The side surfaces ss1 may be connected to the top surface ts at both ends of the top surface ts in the second direction d2. The distance between both side surfaces ss1, in other words, a width w1 of the inner channel 21 in the second direction d2 may be 1.5 times or more and 3 times or less the width of each of the sensors 19 to be described later. When the width w1 is 1.5 times or more, a space in which the fluid sufficiently flows is secured. When the width w1 is 1.5 times or more, the pressure distribution is made uniform and the output of the sensor 19 is stabilized. When the width w1 is 1.5 times or more, a decrease in the flow rate can be reduced. When the width w1 is 3 times or less, an unnecessary increase in the size of the sensor package 10 is suppressed. When the width w1 is 3 times or less, a decrease in flow rate can be reduced. In the present embodiment, the width w1 of the inner channel 21 is twice the width of the sensor 19.

In the main portion 22, a step portion 23 extending in the first direction d1 may be formed on at least one side surface ss1. In the present embodiment, step portions 23 are formed on both side surfaces ss1. Step electrodes 24 for electrically connecting to the sensor 19 may be provided on surfaces s1 of the step portions 23 facing the top surface ts. The height of the step portion 23 from the bottom surface bs may be equal to or greater than the height of the sensor 19 to be described later. A width w2 between the step portions 23 formed on both side surfaces ss1 in the second direction d2 may be 1.1 times or more and 1.5 times or less the width of the sensor 19 to be described later. When the width w2 is 1.1 times or more, a space in which the fluid sufficiently flows is secured. When the width w2 is 1.1 times or more, the pressure distribution is made uniform and the output of the sensor 19 is stabilized. When the width w2 is 1.5 times or less, an unnecessary increase in size of the sensor package 10 is prevented. Since the surfaces of the sensor 19 and the step portions 23 facing the top surface ts in the second direction d2 are continuous, a space for reducing a decrease in the flow rate is secured.

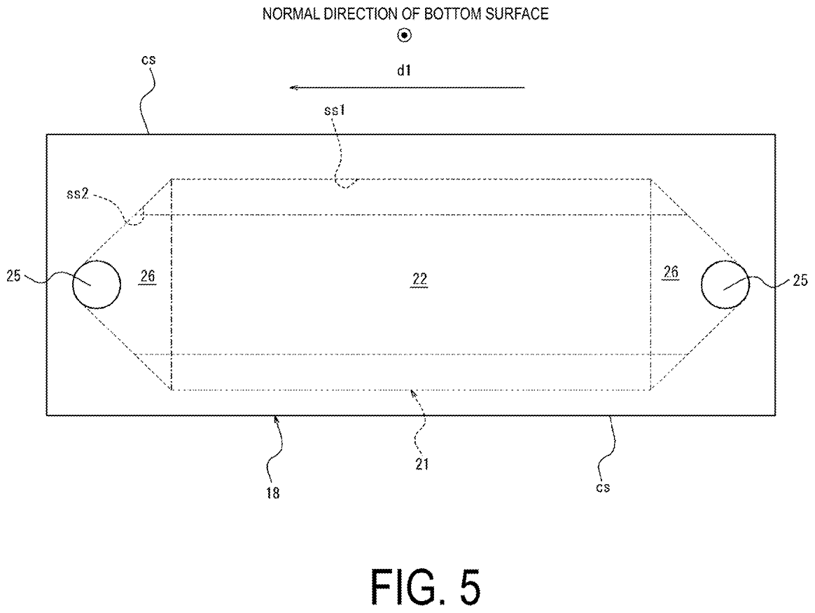

As illustrated in FIG. 5, both ends of the inner channel 21 in the first direction d1 may have a tapered shape with increasing distance from the center of the inner channel 21 when viewed from the normal direction of the bottom surface bs. In the container 18, inflow/outflow openings 25 may be formed in the vicinity of the respective tips of the tapered shapes at both ends. One of the inflow/outflow openings 25 may function as an inflow opening for fluid into the inner channel 21. The other of the inflow/outflow openings 25 may function as an outflow opening for fluid from the inner channel 21. By connecting the main portion 22 to inflow/outflow portions 26 at both ends of the main portion 22 in the first direction d1, the inner channel 21 may have the above-described tapered shape.

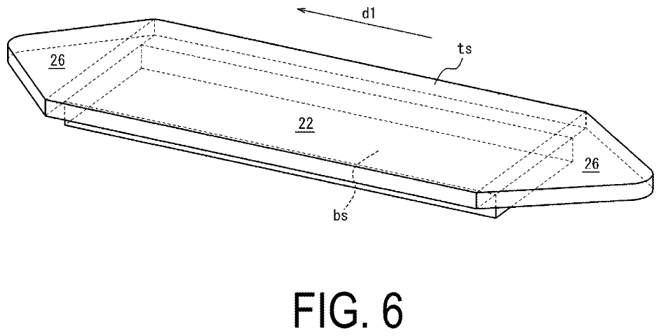

The inflow/outflow portions 26 may have the same bottom surface bs and top surface ts as the main portion 22. As illustrated in FIG. 6, the inflow/outflow portions 26 may include the same top surface ts as the main portion 22 and include a bottom surface that is parallel to the bottom surface bs of the main portion 22 and closer to the top surface ts. The bottom surface may be continuous with a surface of the step portion 23 facing the top surface ts. As illustrated in FIG. 5, the inflow/outflow portions 26 may each include a side surface ss2 that is bent or curved inwardly in the second direction d2 from the side surface ss1 of the main portion 22. The inflow/outflow portions 26 may have a line-symmetric shape with respect to a straight line extending in the first direction d1 when viewed from the normal direction of the bottom surface bs. The inflow/outflow portions 26 may have a substantially isosceles triangular shape that communicates with the main portion 22 at the base when viewed from the normal direction of the bottom surface bs. In the present embodiment, the inflow/outflow portions 26 each have a substantially right-angled isosceles triangular shape when viewed from the normal direction of the bottom surface bs.

An angle between both side surfaces ss2 of each of the inflow/outflow portions 26 may be 60° or more and 120° or less. When the angle between both side surfaces ss2 of each of the inflow/outflow portions 26 is 60° or more, an increase in the size of the sensor package 10 is suppressed. When the angle between both side surfaces ss2 of each of the inflow/outflow portions 26 is 120° or less, the fluid flowing into the inner channel 21 can gradually spread in the second direction d2 toward the main portion 22, which can contribute to equalization of the flow rate and the internal pressure in the second direction d2.

As illustrated in FIG. 3, each of the inflow/outflow openings 25 may be defined by a tubular inner peripheral wall surface perpendicular to the bottom surface bs. The two inflow/outflow openings 25 may be positioned on the top surface ts. The two inflow/outflow openings 25 penetrate to the first surface os1 which is the back surface of the top surface ts. In other words, the two inflow/outflow openings 25 are formed in the first surface os1.

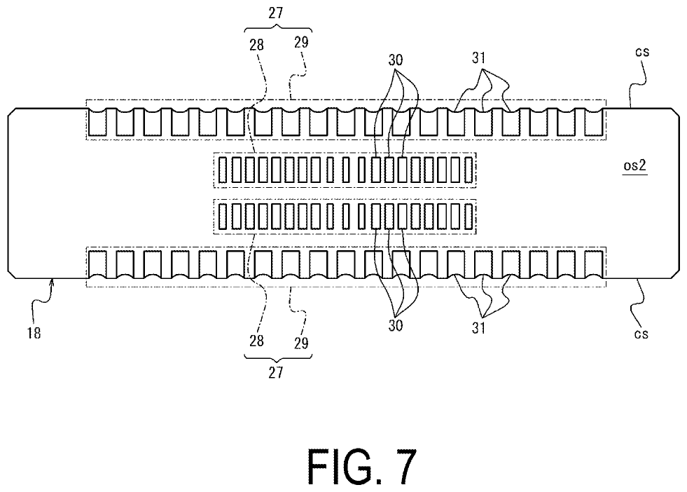

As illustrated in FIG. 7, an electrode group 27 is provided on a surface different from the first surface os1 in the container 18. The surface different from the first surface os1 may be a surface that is discontinuous with the first surface os1. The electrode group 27 may include a first electrode group 28 and a second electrode group 29. The first electrode group 28 may be provided on the second surface os2. The second electrode group 29 may be provided at least on a surface between the first surface os and the second surface os2, for example, on the container side surface cs. The second electrode group 29 may be provided over the second surface os2 and the container side surface cs.

Electrodes 30 constituting the first electrode group 28 may be positioned side by side along the first direction d1. Electrodes 31 constituting the second electrode group 29 may be arranged along the first direction d1. The electrodes constituting the second electrode group 29 may be arranged to be wider than the electrodes constituting the first electrode group 28.

The electrode group 27 is connected to the plurality of step electrodes 24. As will be described later, since the step electrodes 24 are connected to a sensor electrode, the electrode group 27 is electrically connected to the plurality of sensors 19. As illustrated in FIG. 3, each step electrode 24 is connected to a corresponding one of the electrodes 30 in the first electrode group 28 and a corresponding one of the electrodes 31 in the second electrode group 29. For example, with such a configuration, the detection of the plurality of sensors 19 may be output from both the first electrode group 28 and the second electrode group 29.

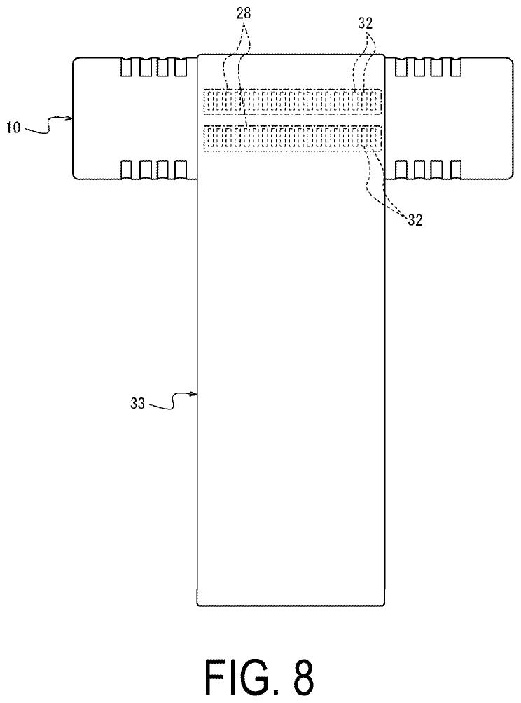

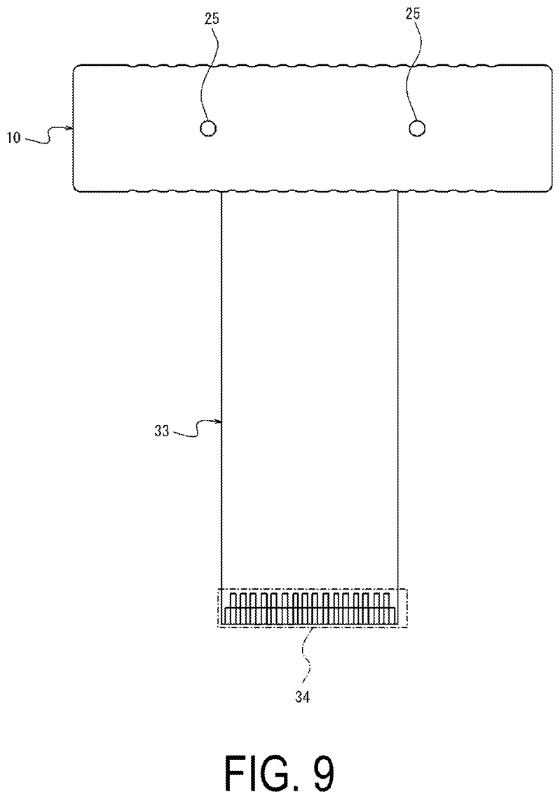

As illustrated in FIG. 8, the first electrode group 28 may be connectable to first terminals (FPC terminals) 32 of Flexible Printed Circuits (FPC) 33. The first terminals 32 are provided on the FPC 33. The first terminal 32 may be a terminal for connection to each electrode 30 in the first electrode group 28. The first electrode group 28 may be connected to the first terminals 32 by soldering, for example. The first electrode group 28 may be connected to the first terminals 32 by an anisotropic conductive paste or an anisotropic conductive film. As illustrated in FIG. 9, the FPC 33 may include a second terminal 34 for detachably connecting to a connector 49 of the sensor module 11.

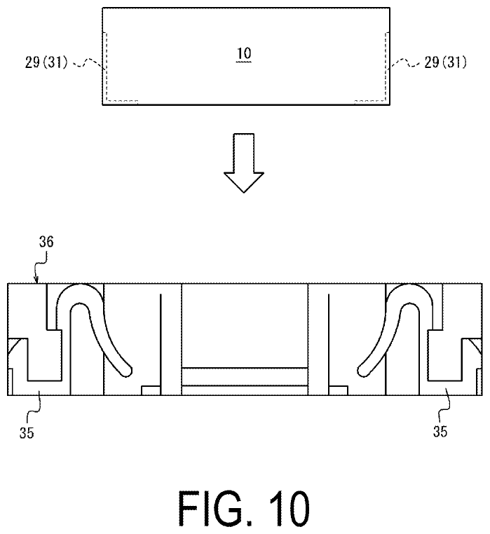

As illustrated in FIG. 10, the second electrode group 29 may be connectable to socket terminals 35. The socket terminals 35 may be terminals provided in a socket 36 for connection to a corresponding one of the electrodes 31 in the second electrode group 29. The socket 36 may detachably mate with the sensor package 10. By fitting the sensor package 10 into the socket 36, each electrode 31 in the second electrode group 29 may be connected to a corresponding one of the socket terminals 35.

As illustrated in FIG. 2, the container 18 may be constituted by a body portion 37 and a cover portion 38. The body portion 37 may include a recess defined by the bottom surface bs and both side surfaces ss1 of the main portion 22 and the bottom surface bs and both side surfaces ss2 of the inflow/outflow portion 26. The inflow/outflow openings 25 may be formed in the cover portion 38. The inner channel 21 may be formed by covering the recess of the body portion 37 with the cover portion 38.

Each of the sensors 19 detects a detection target component in the fluid.

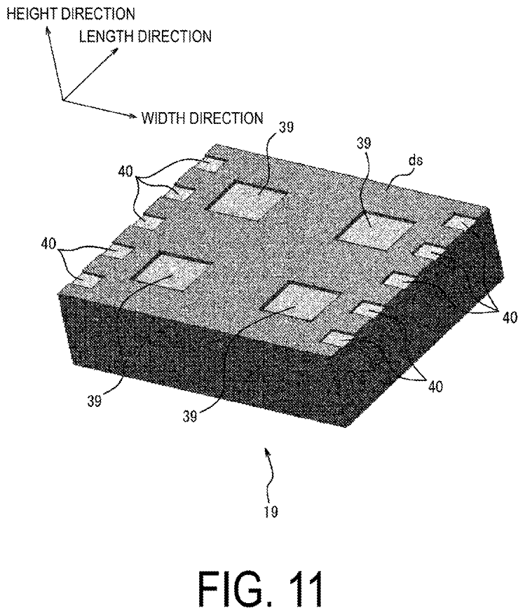

In the sensor 19, a length direction, a width direction, and a height direction may be defined. As illustrated in FIG. 11, the sensor 19 may have a rectangular parallelepiped shape including a flat surface in a combination of two directions among the length direction, the width direction, and the height direction. A detector 39 and a sensor electrode 40 may be provided on a surface on one end side of the sensor 19 in the height direction. Hereinafter, the surface on which the detector 39 and the sensor electrode 40 are provided is referred to as a detection surface ds.

The sensor electrode 40 may be positioned in the vicinity of at least one end or both ends of the sensor 19 in the width direction on the detection surface ds. A plurality of detectors 39 may be provided and may be disposed to be aligned along the length direction and the width direction. In the present embodiment, the sensor 19 is provided with a plurality of detectors 39 arranged along the length direction and the width direction. The sensor 19 may have equal lengths in the length direction and the width direction. The sizes of the plurality of sensors 19, in other words, the lengths in the length direction, the width direction, and the height direction may be equal to each other.

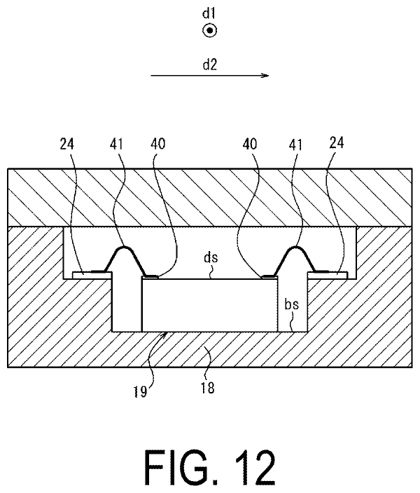

The plurality of sensors 19 may be positioned to be arranged along the first direction d1 in the inner channel 21 of the container 18. The plurality of sensors 19 may be fixed to be positioned on the bottom surface bs. As illustrated in FIG. 12, in the present description, being positioned on the bottom surface bs means that the back surface of the detection surface ds of the sensor 19 is in contact with the bottom surface bs. The sensor 19 may be provided on the bottom surface bs such that the length direction is parallel to the first direction d1 and the width direction is parallel to the second direction d2.

The sensor electrode 40 may be connected to the step electrode 24 positioned in the second direction d2 with respect to the sensor electrode 40 by using a connection wiring line 41. The distance between the two sensors 19 adjacent to each other in the first direction d1 is preferably 0.1 times or more and 1.0 times or less the length of the sensor 19. When the distance is 0.1 times or more, the retention of the fluid between the sensors 19 can be suppressed to shorten the replacement time of the fluid in the inner channel 21, and to secure a space for a mounting margin of the sensor 19. When the distance is 1.0 times or less, a decrease in flow rate is reduced, and an unnecessary increase in the size of the sensor package 10 is suppressed.

The detector 39 has, for example, a film shape. The detector 39 may react particularly strongly to certain components. At least one of the detectors 39 in the plurality of sensors 19 reacts particularly strongly to a first component which is the detection target component. That is, at least one of the detectors 39 in the plurality of sensors 19 detects the detection target component in the fluid. For example, the detector 39 outputs a signal by adsorbing a specific component contained in the fluid. The detector 39 is made of, for example, a polymer material such as polystyrene, chloroprene rubber, polymethyl methacrylate, or nitrocellulose, a semiconductor material such as tin oxide or indium oxide, or the like. The detector 39 outputs a signal corresponding to a reaction with a specific component. This signal is output as a voltage value, for example.

As illustrated in FIG. 2, the heater 20 may heat the inner channel 21 and the sensor 19. The heater 20 may be internally layered in the container 18. The heater 20 may be positioned on the bottom surface bs side of the inner channel 21. In the present embodiment, the heater 20 is internally layered in the body portion 37. The heater 20 is, for example, a high-resistance metal heater or a ceramic heater.

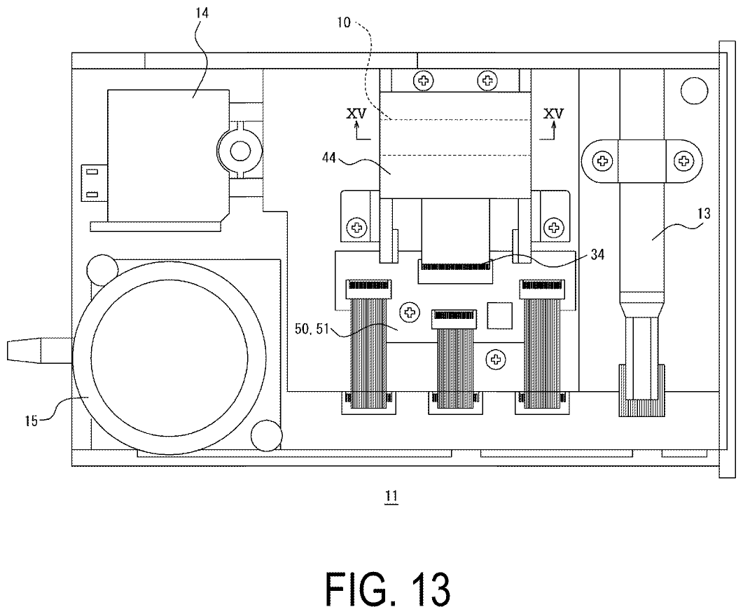

As illustrated in FIG. 13, the sensor package 10 can be detachably fixed to the sensor module 11 by a fixing portion 44. By fixing the sensor package 10 to the sensor module 11, the channel 16 and the inflow/outflow openings 25 may be connected to each other. The sensor package 10 and a controller 50 may be connected to each other by connecting the second terminal 34 to a connector 49 which will be described later.

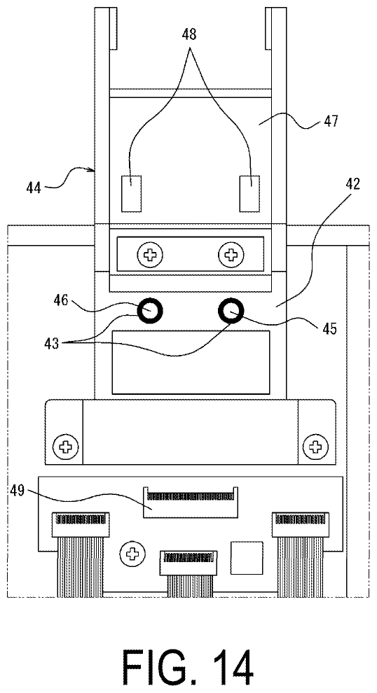

As illustrated in FIG. 14, the sensor module 11 may include a package placement surface 42, sealing bodies 43, and a fixing portion 44 for fixing the sensor package 10 so as to connect the channel 16 of the sensor module 11 and the inflow/outflow openings 25 of the sensor package 10. The sensor module 11 may further include the connector 49 for electrically coupling with the second terminal 34.

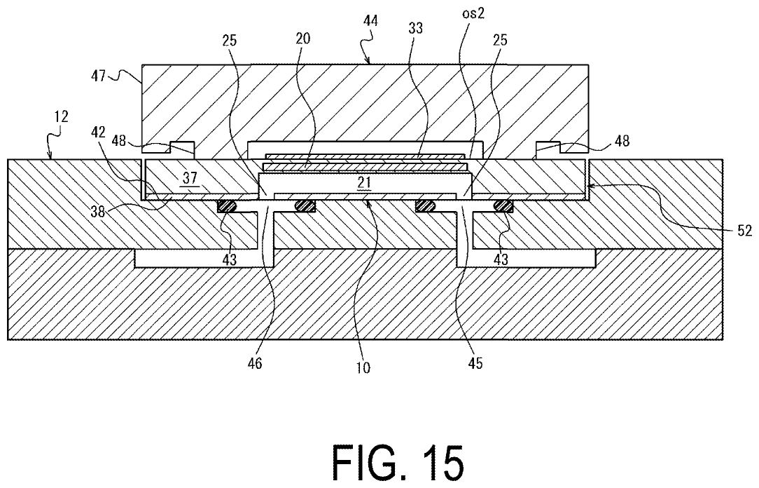

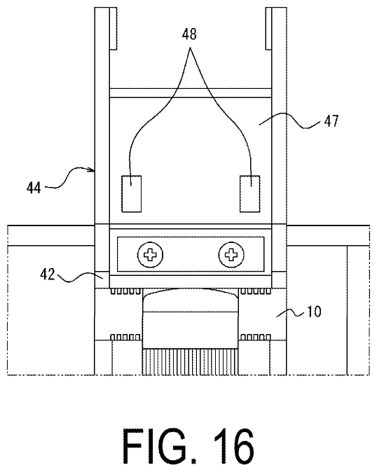

As illustrated in FIG. 15, the package placement surface 42 may be a bottom surface of a recessed portion 52 recessed from a flat surface of a part of the housing 12. The recessed portion 52 may be shaped to fit the sensor package 10. The package placement surface 42 may be provided with a supply port 45 and a discharge port 46 for enabling fluid to flow to the sensor package 10. As illustrated in FIGS. 15 and 16, the sensor package 10 may be placed on the package placement surface 42 so that the sensor package 10 and the recessed portion 52 are fitted to each other. For example, the placement position and the placement posture of the sensor package 10 on the package placement surface 42 may be determined in advance as the normal position and the normal posture in a state where the sensor package 10 is fitted in the recessed portion 52. When the sensor package 10 is placed in the normal position and the normal posture, the inflow/outflow opening 25 functioning as the inflow opening and the supply port 45s may be connected to each other, and the inflow/outflow opening 25 functioning as the outflow opening and the discharge port 46 may be connected to each other.

As illustrated in FIGS. 14 and 15, the sealing bodies 43 may be provided at the supply port 45 and the discharge port 46. Each of the sealing bodies 43 may be, for example, an annular elastic body such as an O-ring. One of the sealing bodies 43 may seal a connecting portion between the inflow/outflow opening 25 functioning as the inflow opening and the supply port 45. The other of the sealing bodies 43 may seal a connecting portion between the inflow/outflow opening 25 functioning as the outflow opening and the discharge port 46.

As illustrated in FIG. 13, the fixing portion 44 may fix the sensor package 10 by pressing the sensor package 10 against the package placement surface 42. By opening the fixing portion 44, the sensor package 10 may be removed from the sensor module 11.

As illustrated in FIG. 15, the fixing portion 44 may include a plate-like portion 47. The plate-like portion 47 may be pivotally supported about a straight line parallel to the package placement surface 42, and may be openable and closable with respect to the package placement surface 42. The plate-like portion 47 may include a step portion 48 on a surface facing the package placement surface 42 in a state where the plate-like portion 47 is closed.

The step portion 48 may press a part of the second surface os2 in a state where the sensor package 10 is placed on the package placement surface 42 in the normal position and the normal posture. The part of the second surface os2 may be, for example, a region outside the first electrode group 28. The part of the second surface may be a region outside the FPC 33.

The sensor module 11 may include at least one of the connector 49 and the socket 36 for indirect connection to the electrode group 27 of the sensor package 10.

For example, as illustrated in FIG. 14, the sensor module 11 may include the connector 49 for detachably connecting to the second terminals 34 of the FPC 33 connected to the first electrode group 28 of the sensor package 10. The connector 49 may be connected to the controller 50, which will be described later. By connecting the second terminals 34 to the connector 49, the sensor package 10 may be electrically connected to the controller via the FPC 33. In other words, the sensor 19 may be electrically connected to the controller 50 via the connection wiring line 41, the step electrode 24, the first electrode group 28, the FPC 33, and the connector 49.

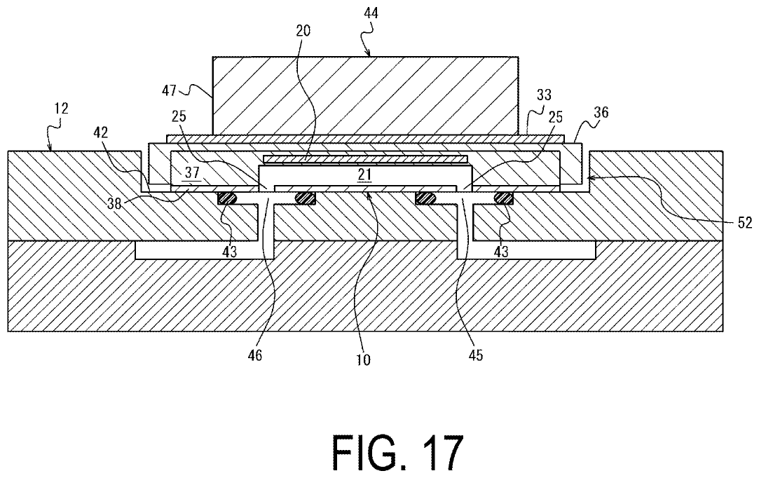

For example, as illustrated in FIG. 17, the sensor module 11 may include the socket 36 including the socket terminal 35 for connection to the second electrode group 29 of the sensor package 10. The socket 36 may be provided on a surface of the plate-like portion 47 that faces the package placement surface 42 in a state where the plate-like portion 47 is closed, instead of the step portion 48.

The sensor package 10 and the channel of the sensor module 11 may be connected to each other by placing the socket 36 on the package placement surface 42 and the bottom surface of the recessed portion 52 in an upward direction, inserting the sensor package 10 into the socket 36, and fixing the sensor package 10 with the plate-like portion 47, so that the sensor package 10 and the controller 50 may be electrically connected to each other via the socket 36.

The socket terminal 35 may be connected to the first terminal 32 of the FPC 33 instead of the first electrode group 28. By inserting the sensor package 10 into the socket 36 and connecting the second terminal 34 of the FPC 33 to the connector 49, the sensor package 10 may be electrically connected to the controller via the socket 36 and the FPC 33. In other words, the sensor 19 may be electrically connected to the controller 50 via the connection wiring line 41, the step electrode 24, the first electrode group 28, the FPC 33, and the connector 49.

The socket terminal 35 may be directly connected to the controller 50 described later. In a configuration in which the socket terminal 35 is directly connected to the controller, the sensor package 10 may be electrically connected to the controller via the socket 36 by inserting the sensor package 10 into the socket 36. In other words, by inserting the sensor package 10 into the socket 36, the sensor 19 may be electrically connected to the controller 50 via the socket 36.

In FIG. 1, the measurement unit 14 may include a sensor capable of measuring a predetermined property or condition related to the fluid supplied to the sensor module 11. The predetermined property or condition related to the fluid may be a property or condition that may affect the detection accuracy of the fluid in the sensor package 10. The predetermined property or condition related to the fluid may include, for example, one of the temperature and humidity of the fluid. In this description, the predetermined property or condition of the fluid will be described below as being the temperature and humidity of the fluid. In this case, the measurement unit 14 may include, for example, a thermo-hygrometer. The thermo-hygrometer may measure the temperature and humidity of the fluid in a conventionally known manner. The signal of the detector 39 can be corrected based on the temperature and humidity of the fluid measured by the measurement unit 14. However, the sensor module 11 may not necessarily include the measurement unit 14. The sensor module 11 can calculate the concentration of the detection target component without including the measurement unit 14.

The pump unit 15 may draw the fluid supplied to the sensor module 11 from the upstream side to the downstream side and discharge the fluid to the outside of the sensor module 11. That is, the fluid supplied from the first channel 17a or the second channel 17b to the sensor module 11 by the suction of the pump unit 15 passes through the switching unit 13, the sensor package 10, the measurement unit 14, and the pump unit 15, and is discharged to the outside of the sensor module 11 via the third channel 17c. The pump unit 15 can control the drawing amount of the fluid. For example, the flow rate of the fluid flowing through the channel 16 is controlled by controlling the drawing amount of the fluid by the pump unit 15.

For example, the pump unit 15 may control the drawing amount of the fluid to suppress a change in the flow rate of the fluid in the channel 16. The pump unit 15 may include, for example, a piezo pump. The pump unit 15 may include one pump. The pump unit 15 may include a plurality of pumps. In this case, the plurality of pumps may be disposed in parallel with respect to the fluid flow.

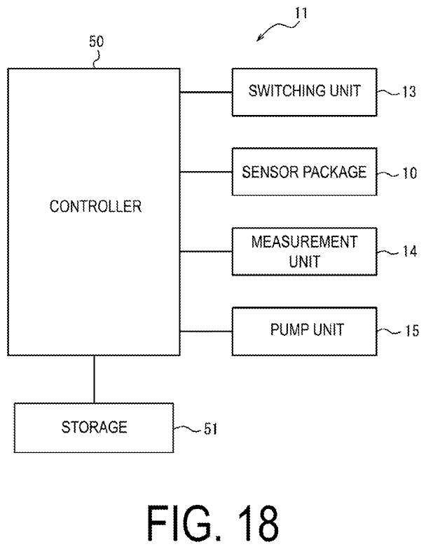

As illustrated in FIG. 18, the sensor module 11 may further include an electronic circuit substrate in the housing 12. The electronic circuit substrate may mount the controller 50, a storage 51, and the like of the sensor module 11, which will be described later.

FIG. 18 is a functional block diagram illustrating an overall configuration of the sensor module 11 in FIG. 1. The sensor module 11 in FIG. 18 may include the controller 50, the storage 51, the switching unit 13, the sensor package 10, the measurement unit 14, and the pump unit 15.

The switching unit 13 may receive a control signal from the controller 50 and perform switching between the first channel 17a and the second channel 17b based on the control signal. Accordingly, either the test fluid or the control fluid may be supplied to the channel 16.

The sensor package 10 may transmit and receive input and output signals to and from each sensor 19 to and from the controller 50.

The measurement unit 14 may transmit and receive a signal of measured information to and from the controller 50.

The pump unit 15 may receive a control signal from the controller 50. The pump unit 15 may draw the fluid to the downstream side on the basis of the control signal. The pump unit 15 may draw the fluid by a drawing amount corresponding to the control signal.

The controller 50 is, for example, a processor configured to control and manage the entirety of the sensor module 11 including each functional block of the sensor module 11. The controller 50 may be constituted by a processor such as a central processing unit (CPU) configured to execute a program defining a control procedure. Such a program may be stored in the storage 51, an external storage medium connected to the sensor module 11, or the like, for example.

The controller 50 may calculate the concentration of the detection target component in the test fluid on the basis of the signal output from the sensor package 10. The controller 50 may further calculate the concentration of the detection target component in the test fluid based on the signal output from the measurement unit 14. Depending on the nature or condition of the fluid, the reactivity of the detection target component in each sensor 19 of the sensor package 10 may change. When calculating the concentration of the detection target component in the test fluid on the basis of the signal output from the measurement unit 14 as described above, the controller 50 can calculate the concentration of the detection target component in consideration of reactivity. Therefore, the calculation accuracy of the concentration of the detection target component can be improved.

The controller 50 may convert analog signals output from the plurality of sensors 19 and the measurement unit 14 into digital data. The controller 50 may store the converted digital data in the storage 51. The controller 50 may be controlled by a control device such as a personal computer which is an external device of the sensor module 11.

The storage 51 may be constituted by a semiconductor memory, a magnetic memory, or the like. The storage 51 stores various kinds of information and/or a program for operating the sensor module 11. The storage 51 may function as a working memory.

Next, the control of the switching unit 13 and the calculation of the concentration of the detection target component by the controller 50 will be described in detail.

A test fluid (sample gas) is supplied to the first channel 17a. Here, as an example, a case where the test fluid is human exhaled breath will be described. However, the test fluid is not limited to human exhaled breath and may be any fluid to be tested. When the test fluid is human exhaled breath, the detection target component is, for example, acetone, ethanol, carbon monoxide, or the like. The detection target component is not limited to the examples given here. The test fluid contains a noise component (noise gas) which is a second component. The noise component is a component other than the detection target component. The noise component includes all components other than the detection target component, such as oxygen, carbon dioxide, nitrogen, and water vapor.

A control fluid (refresh gas) is supplied to the second channel 17b. The control fluid may be, for example, a fluid that does not substantially contain the detection target component. Here, “does not substantially contain the detection target component” includes cases where the detection target component is not contained at all as well as cases where the content of the detection target component in the control fluid is so small with respect to the content of the detection target component in the test fluid that it can be considered to be practically not contained. When the test fluid is human exhaled breath, air can be used as the control fluid, for example. However, the control fluid is not limited to air. The control fluid contains noise components such as oxygen, carbon dioxide, nitrogen and water vapor.

The controller 50 sets the drawing amount of the pump unit 15 to be constant, and switches the switching unit 13 between the first channel 17a and the second channel 17b at certain time intervals. The certain time interval may be determined to be appropriate in accordance with, for example, the type or property of the test fluid. Here, an example will be described where the certain time interval is set to 5 seconds. Therefore, the controller 50 controls the switching unit 13 to switch the channel connected to the channel 16 between the first channel 17a and the second channel 17b every 5 seconds.

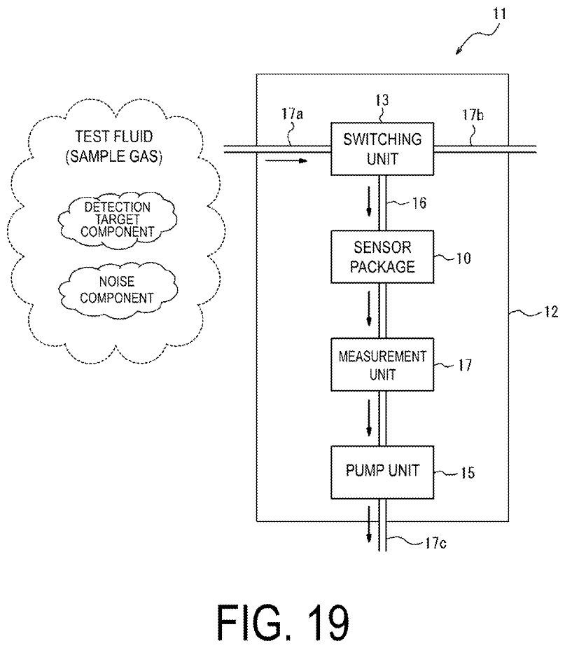

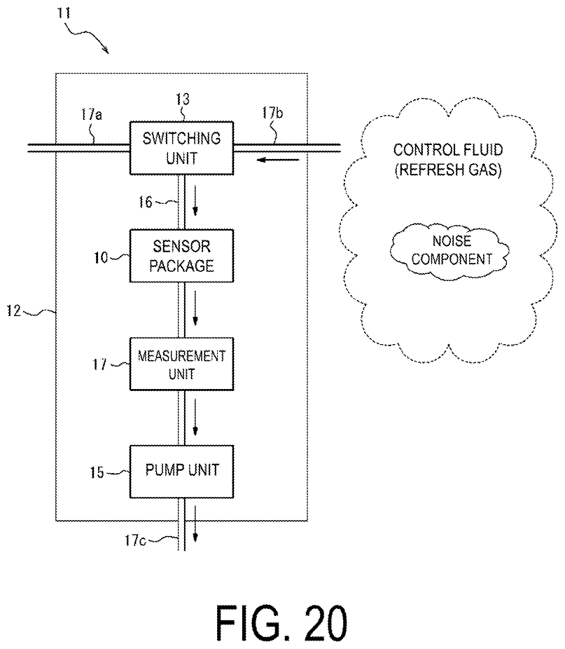

FIGS. 19 and 20 are diagrams schematically illustrating an example of fluid flow. FIG. 19 illustrates an example in which the first channel 17a is connected to the channel 16. FIG. 20 illustrates an example in which the second channel 17b is connected to the channel 16. That is, in the example described here, the state depicted in FIG. 19 and the state depicted in FIG. 20 are alternately repeated every 5 seconds. The arrows in FIGS. 19 and 20 indicate the direction of fluid flow.

As illustrated in FIG. 19, when the first channel 17a is connected to the channel 16, the test fluid is supplied from the first channel 17a to the sensor package 10 by drawing of the pump unit 15. In this case, each detector 39 in each sensor 19 of the sensor package 10 reacts with a component contained in the test fluid. Each sensor 19 outputs a signal (first signal) corresponding to a component of the test fluid including the detection target component and the noise component.

As illustrated in FIG. 20, when the second channel 17b is connected to the channel 16, the control fluid is supplied from the second channel 17b to the sensor package 10 by drawing of the pump unit 15. In this case, each sensor 19 of the sensor package 10 reacts with a component contained in the control fluid. Each sensor 19 outputs a signal (second signal) corresponding to a component of the control fluid including the noise component.

The first signal and the second signal are signals supplied to the controller 50 by the sensor package 10 reacting with the test fluid and the control fluid, respectively. Both the test fluid and the control fluid contain the noise component. Therefore, both the first signal and the second signal reflect the same reactivity with respect to the noise component contained in the fluid supplied to the sensor package 10.

On the other hand, regarding the detection target component, the test fluid contains the detection target component, whereas the control fluid does not substantially contain the detection target component. Therefore, it can be said that the first signal is a signal in which the reactivity with respect to the detection target component is reflected, whereas the second signal is a signal in which the reactivity with respect to the detection target component is not substantially reflected. Therefore, the difference between the first signal and the second signal output from the sensor package 10 can be considered to be substantially the concentration of the detection target component contained in the test fluid. The controller 50 can calculate the concentration of the detection target component based on the difference.

The sensor package 10 of the present embodiment configured as described above includes the inner channel 21 in which the plurality of sensors 19 are provided and through which the fluid flows and the container 18 including the inflow opening to the inner channel 21 and the first surface os1 on which the outflow opening from the inner channel 21 is formed, and the electrode group 27 electrically connected to the plurality of sensors 19 is provided on a surface different from the first surface os1 of the container 18. With such a configuration, in the sensor package 10, the inflow/outflow openings 25 functioning as an inflow opening and an outflow opening and the electrode group 27 can be easily connected to the sensor module 11. Therefore, the sensor package 10 can be easily replaced from the sensor module 11.

In the sensor package 10 of the present embodiment, the electrode group 27 includes the first electrode group 28 and the second electrode group 29 both of which can output detection of the plurality of sensors 19. The first electrode group 28 is provided on the back surface (second surface os2) of the first surface os1, and the second electrode group 29 is provided on a surface between the first surface os1 and the back surface. With such a configuration, the sensor package 10 can be electrically connected to the sensor module 11 including either the first terminal 32 or the socket terminal 35.

The sensor module 11 of the present embodiment includes a sealing bodies 43 configured to respectively seal a connecting portion between the inflow/outflow opening 25 functioning as an inflow opening and the supply port 45 and a connecting portion between the inflow/outflow opening 25 functioning as an outflow opening and the discharge port 46, and a fixing portion 44 configured to detachably fix the sensor package 10 to the package placement surface 42 thereby pressing the sensor package 10 against the package placement surface 42. According to such a configuration, in the sensor module 11, the sensor package 10 can be fixed in a state in which the inflow/outflow opening 25 functioning as the inflow opening and the supply port 45 and the inflow/outflow opening 25 functioning as the outflow opening and the discharge port 46 are easily connected.

In the sensor module 11 of the present embodiment, the fixing portion 44 includes the plate-like portion 47 that can be opened and closed. With such a configuration, in the sensor module 11, the fixing portion 44 can be easily opened and closed, and thus the sensor package 10 can be easily attached and detached.

In the sensor module 11 of the present embodiment, the recessed portion 52 to be fitted to the sensor package 10 is formed. With such a configuration, the sensor module 11 can easily align the sensor package 10.

In the sensor module 11 of the present embodiment, the plate-like portion 47 has the step portion 48 capable of pressing the sensor package 10 in a part of the back surface (second surface os2) of the first surface os1, for example, in a region outside the FPC 33. Since the FPC generally undulates, stably pressing the sensor package 10 against the package placement surface 42 by pressing via the FPC 33 is difficult, and the sealing performance of the supply port 45 and the inflow opening and the sealing performance of the discharge port 46 and the outflow opening may deteriorate. On the other hand, since the sensor module 11 having the above-described configuration can press the sensor package 10 at a position outside the FPC 33, the sensor package 10 can be stably pressed.

The sensor package 10 of the present embodiment includes the container 18 including the inner channel 21 through which the fluid flows along the first direction d1, and the plurality of sensors 19 which are positioned in the inner channel 21 such that they are arranged along the first direction d1 and detect the detection target component in the fluid. With such a configuration, the sensor package 10 can reduce the retention of the fluid in the inner channel 21 on the whole and shorten the retention time. Therefore, the sensor package 10 can improve the responsiveness of each sensor 19 after the fluid flows into the sensor package 10. Since the responsiveness of each sensor 19 is improved, the sensor package 10 can detect the odor caused by the combination of the detection target components of the plurality of sensors 19 with high detection accuracy. In the sensor package 10 having the above-described configuration, the pressure in the inner channel 21 is equalized, thus reducing a detection error due to a difference in pressure to the plurality of sensors 19. Therefore, the sensor package 10 can further improve the odor detection accuracy.

In the sensor package 10 of the present embodiment, the plurality of sensors 19 are positioned on the flat bottom surface bs. With such a configuration, in the sensor package 10, the bottom surface bs and the detection surface ds of the sensor 19 do not form a continuous flat surface but form a recessed step with respect to the detection surface ds. Due to this step, the flow rate of the fluid between the detection surface ds and the top surface ts can be homogenized. Such an action is not theorized, but is presumed to be as follows. Since the fluid has viscosity, the flow rate of the fluid flowing along a surface whose entire surface is flat is considered to decrease in the vicinity of the surface. On the other hand, as in the sensor package 10 described above, in the fluid flowing along the surface including the recessed step with respect to the detection surface ds, it is considered that a decrease in flow rate in the vicinity of the surface on which the detector 39 is formed is suppressed by the recessed step, and the flow rate of the fluid between the detection surface ds and the top surface ts can be homogenized.

In the sensor package 10 of the present embodiment, both ends of the inner channel 21 in the first direction d1 have a tapered shape with increasing distance from the center of the inner channel 21 when viewed from the normal direction of the bottom surface bs, and the inflow/outflow opening 25s are formed in the container 18 in the vicinity of the tips of the tapered shapes at both ends of the inner channel 21. With such a configuration, the sensor package 10 can equalize the pressure and the concentration of the fluid in the second direction d2 of the inner channel 21. Therefore, the sensor package 10 can further improve the odor detection accuracy.

In the sensor package 10 of the present embodiment, each of the inflow/outflow openings 25 is defined by a tubular inner peripheral wall surface perpendicular to the bottom surface bs. With such a configuration, the sensor package 10 can cause the fluid flowing into the inner channel 21 from the inflow/outflow opening 25 to collide with the bottom surface bs, and thus can equalize the pressure in the entire region of the inner channel 21. With such a configuration, in the sensor package 10, the fluid easily flows into the space surrounded by the side surface of the sensor 19, the side surface of the step portion 23, and the bottom surface bs. With such a configuration, in the sensor package 10, the fluid easily flows into the space surrounded by the space between the sensors 19 and the bottom surface bs. As a result, the sensor package 10 can suppress retention of the fluid and increase the flow rate of the fluid along the first direction d1.

In the sensor package 10 of the present embodiment, the container 18 includes the body portion 37 including a recess and the cover portion 38 in which the inflow/outflow openings 25 are formed, and the inner channel 21 is formed by covering the recess with the cover portion 38. With such a configuration, the sensor package 10 can be manufactured by a simple method.

In the sensor package 10 of the present embodiment, the container 18 is made of ceramic. With such a configuration, the sensor package 10 can suppress a component of the container 18 from being mixed into the fluid due to liquefaction, vaporization, or the like of the body of the container 18. Therefore, the sensor package 10 can suppress deterioration in the detection accuracy of the detection target component.

The sensor package 10 of the present embodiment includes the heater 20. Therefore, in the sensor package 10, by heating the inner channel 21 and the sensor 19 using the heater 20, the fluid adsorbed to the inner channel 21 and the sensor 19 is desorbed, and the inner channel 21 can be refreshed. In the sensor package 10, since the temperature fluctuation in the inner channel 21 is reduced by the heater 20, deterioration in the detection accuracy of the detection target component can be suppressed regardless of the temperature change of the test fluid. By changing the temperature of the sensor 19 by the heater 20 to change the detection sensitivity and selectivity of the sensor 19, the detection accuracy of the detection target component can be improved.

In the sensor package 10 of the present embodiment, the step portions 23 extending in the first direction d1 are formed on both sides of the bottom surface bs in the second direction d2. With such a configuration, in the sensor package 10, the fluid between the detection surface ds and the top surface ts of the sensor 19 is further collected, the flow rate is increased, and the fluid arrival time is shortened. In the sensor package 10, the connection wiring line 41 is disposed on the side surface ss1 side in the second direction d2 with respect to the detector 39, and thus the fluid can smoothly flow on the detector 39 and the flow rate of the fluid on the detector 39 can be increased. On the other hand, as in the sensor package 10 described above, between the detection surface ds and the top surface ts of the sensor 19, the side surface ss1 is positioned away from the sensor 19 along the second direction d2. Therefore, in the sensor package 10, between the surface on which the detector 39 is formed and the top surface ts, a decrease in flow rate of the fluid around the end portion in the second direction d2 is suppressed, and a difference in flow rate due to a difference in position in the second direction d2 can be reduced. When the entirety of the side surface ss1 is separated from the sensor 19, the flow rate decreases on the whole due to an increase in the volume of the inner channel 21. On the other hand, according to the above-described configuration, in the sensor package 10, the entirety of the side surface ss1 is not separated from the sensor 19, and thus a difference in flow rate in the region contributing to the improvement of the detection accuracy can be reduced while suppressing the decrease in flow rate on the whole.

In the sensor package 10 according to the present embodiment, the height of the step portion 23 with respect to the bottom surface bs is equal to or greater than the height of the sensor 19. With such a configuration, the sensor package 10 allows a large amount of fluid to flow on the sensor 19 by narrowing the space between the step portion 23 and the top surface ts, thereby increasing the flow rate. As a result, the sensor package 10 can shorten the retention time of the fluid and shorten the detection time difference between each detector 39 of each sensor 19, thereby improving the detection accuracy of the detection target component.

In the sensor package 10 of the present embodiment, the sensor electrode 40 and the step electrode 24 are connected by the connection wiring line 41 in order to transmit a signal output from the detector 39 to the outside of the sensor package 10. With this wiring structure, the decrease in the flow rate of the fluid due to the connection wiring line 41 on the sensor 19 is suppressed, and the detection accuracy of the detection target component is improved.

In the sensor module 11 of the present embodiment, the fluid is drawn by the pump unit 15 provided in the channel 16 and the fluid is supplied to the sensor package 10. The fluid supplied to the sensor package 10 is switched between the test fluid and the control fluid by switching between the first channel 17a and the second channel 17b by the switching unit 13. Therefore, regardless of whether the fluid supplied to the sensor package 10 is the test fluid or the control fluid, the fluid is drawn to the downstream side by the same pump unit 15. If the pump that supplies the test fluid to the sensor package 10 and the pump that supplies the control fluid are different from each other, a difference may occur between the supply amount of the test fluid and the supply amount of the control fluid due to a difference in the performance of the pumps or the like. However, in the sensor module 11 of the present embodiment, the fluid supplied to the sensor package 10 is controlled by the single pump unit 15, and thus the fluid can be supplied more stably to the sensor package 10 compared with a case where a different pump is used for each fluid to be supplied. Accordingly, the conditions under which the test fluid and the control fluid are supplied to the sensor package 10 are likely to be equal to each other. Therefore, the sensor package 10 can easily detect the test fluid and the control fluid under more equal conditions. Therefore, according to the sensor module 11, the measurement accuracy of the detection target component can be improved.

Although the present disclosure has been described with reference to the drawings and examples, note that a person skilled in the art can easily make various variations and/or modifications based on the present disclosure. Accordingly, note that these variations and/or modifications are included within the scope of the present invention. For example, the functions and the like included in each of the components and the like can be rearranged as long as they are logically consistent, and a plurality of components can be combined into one or divided.

In the present disclosure, terms “first”, “second”, and the like are identifiers for distinguishing the configurations. Configurations distinguished by the terms “first”, “second”, and the like in the present disclosure can exchange the numbers in the configurations with each other. The identifiers are interchanged simultaneously. The configurations are distinguished even after the identifiers are interchanged. The identifiers may be deleted. Configurations with identifiers deleted are distinguished by reference signs. No interpretation on the order of the configurations, and no grounds for the presence of an identifier of a lower value shall be given based solely on the description of identifiers such as “first” and “second” in the present disclosure.

### REFERENCE SIGNS

10 Sensor package
11 Sensor module
12 Housing
13 Switching unit
14 Measurement unit
15 Pump unit
16 Channel
17a First channel
17b Second channel
17c Third channel
18 Container
19 Sensor
20 Heater
21 Inner channel
22 Main portion
23 Step portion
24 Step electrode
25 Inflow/outflow opening
26 Inflow/outflow portion
27 Electrode group
28 First electrode group
29 Second electrode group
30 Electrode constituting first electrode group
31 Electrode constituting second electrode group
32 First terminal
33 FPC
34 Second terminal
35 Socket terminal
36 Socket
37 Body portion
38 Cover portion
39 Detector
40 Sensor electrode
41 Connection wiring line
42 Package placement surface
43 Sealing body
44 Fixing portion
45 Supply port
46 Discharge port
47 Plate-like portion
48 Step portion
49 Connector
50 Controller
51 Storage
52 Recessed portion
bs Bottom surface
cs Container side surface
d1 First direction
d2 Second direction
ds Detection surface
os1 First surface
os2 Second surface
s1 Surface facing top surface
ss1 Side surface of main portion
ss2 Side surface of inflow/outflow portion
ts Top surface
w1 Width of inner channel
W2 Width of step portions formed on both side surfaces

1. A sensor package comprising:
a plurality of sensors configured to detect a detection target component in a fluid; and
a container comprising an inner channel in which the plurality of sensors are provided and through which a fluid flows, and a first surface on which an inflow opening to the inner channel and an outflow opening from the inner channel are formed, wherein
an electrode group electrically connected to the plurality of sensors is provided on a surface different from the first surface of the container.

2. The sensor package according to claim 1, wherein
the electrode group contains a first electrode group and a second electrode group both of which are configured to output detection of the plurality of sensors,
the first electrode group being provided on a back surface of the first surface, and
the second electrode group being provided on a surface between the first surface and the back surface.

3. The sensor package according to claim 2, wherein
electrodes constituting the first electrode group are arranged in a first direction, and
electrodes constituting the second electrode group are arranged to be wider than the electrodes constituting the first electrode group in the first direction.

4. The sensor package according to claim 2, wherein
the first electrode group is connected to an FPC terminal.

5. The sensor package according to claim 2, wherein
the second electrode group is connected to a socket terminal.

6. A sensor module comprising:
a package placement surface on which a supply port and a discharge port configured to enable a fluid to flow to the sensor package according to claim 1 are provided;
sealing bodies respectively provided at the supply port and the discharge port and configured to seal a connecting portion between the supply port and the inflow opening and a connecting portion between the discharge port and the outflow opening; and
a fixing portion configured to detachably fix the sensor package thereby pressing the sensor package against the package placement surface.

7. The sensor module according to claim 6, wherein
the fixing portion comprises a plate-like portion that can be opened and closed.

8. The sensor module according to claim 7, wherein
the plate-like portion comprises a step portion configured to press a part of a back surface of the first surface in a state where the sensor package is placed on the package placement surface, where the supply port is connected to the inflow opening and the discharge port is connected to the outflow opening.

9. A sensor device comprising:
the sensor module according to claim 6.

10. The sensor package according to claim 3, wherein
the first electrode group is connected to an FPC terminal.

11. The sensor package according to claim 3, wherein
the second electrode group is connected to a socket terminal.
